(19) United States
(12) Patent Application Publication
(10) Pub. No.: US 2008/0113398 A1
(43) Pub. Date: May 15, 2008

(54) Apparatus and method for quantitatively analyzing enzyme inhibitor

(75) Inventors: Yueh-Hui Lin, Hsinchu (TW); Jui-Ping Wang, Hsinchu (TW); Yi-Feng Cheng, Changhua County (TW); Yu-Sen Hsu, Taipei (TW); Ming-Chang Hsu, Hsinchu (TW); Cheng Allen Chang, Hsinchu (TW); Moon Wen Yang, Hsinchu (TW); Thomas Y.S. Shen, Hsinchu (TW)

Correspondence Address: REED SMITH LLP, Suite 1400, 3110 Fairview Park Drive, Falls Church, VA, 22042, US

(73) Assignee: Apex Biotechnology Corp.

(21) Appl. No.: 11/979,620

(22) Filed: Nov. 6, 2007

(30) Foreign Application Priority Data
Nov. 6, 2006 (TW) 95140989

(51) Int. Cl. C12Q 1/48; G06F 19/00; C12M 1/34

(52) U.S. Cl. 435/15; 435/287.2; 702/19

(57) ABSTRACT

A method and an apparatus for quantitatively analyzing an enzyme inhibitor are provided. The apparatus includes a memory unit for storing at least one set of parameters, an interface for receiving a test strip, a reflective type optical detection unit for providing a light beam onto the test strip to obtain a test signal, a processor for processing the test signal based on the at least one set of parameters to obtain a quantitative analysis result, and a display unit for outputting the quantitative analysis result.

### CROSS REFERENCE TO RELATED APPLICATIONS

This application claims the right of priority based on Taiwan Patent Application No. 095140989 entitled “APPARATUS AND METHOD FOR QUANTITATIVE ANALYZING ENZYME INHIBITOR”, filed on Nov. 6, 2006, which is incorporated herein by reference and assigned to the assignee herein.

### FIELD OF THE INVENTION

The present invention relates to an apparatus and a method for detecting an enzyme inhibitor, and more particularly, relates to an apparatus and a method for quantitatively analyzing an enzyme inhibitor.

### BACKGROUND OF THE INVENTION

There are many methods for determining the amount of pesticides remained within the vegetables and fruits in the prior art, such as spectrophotometry, atomic absorption spectrometry, thin-layer chromatography, gas chromatography, liquid chromatography, NMR spectroscope, fluorescence, immunoassay, etc. Among these methods, the gas chromatography and the liquid chromatography are most common because they are good in reproducibility and sensitivity, and moreover, in determining the type of pesticides. However, these methods need a lot of samples prepared by professional technicians in laboratories, and the untrained users can not easily and quickly determine the amount or the type of residual pesticides in a shopping place, such as a crop filed, a farmers' market or a supermarket.

In recent years, biochemical reaction and electrochemical techniques have been used in determining the type of enzyme inhibitors within pesticides. For example, the colorimetric analysis can be applied to determine the presence of the enzyme inhibitor. In a reaction basin, acetylcholinesterase (AChE), butylcholinesterase (BchE), or cholinesterase (ChE) is reacted with a sample, which may contain enzyme inhibitor, and then color indicator is added to determine the percentage of the enzyme inhibited due to the presence of the enzyme inhibitor by a spectrophotometer. However, this kind of technique creates lots of wastes required of careful treatments, which is highly inconvenient and troublesome for the operator.

In order to reduce the wastes, a test strip is developed. For a conventional test strip, the sample is provided to an enzyme test pad, and then the test strip is folded by fingers so that the test pad can react with a color indicator. The user must hold the folded test strip for a certain period of time till the reaction is achieved, and then unfold the test strip inspecting the color of the color indicator by the naked eye to determine whether the enzyme inhibitor exists in the sample.

As known, the activity of the enzyme is significantly affected by temperature. A heating device is therefore provided in incorporation with the test strip. However, the heating device has some drawbacks, such as sample leaking out, test strip positioning difficulty, the enzyme test pad not fully contacting the color indicator, or long time heating process causing the sample evaporated. Furthermore, the process takes longer time and the operator may directly expose to the extract of the pesticides danger to his/her health.

Therefore, there is a need to provide a novel apparatus and a method to overcome the limitations, and enable unprofessional users to conveniently perform the test qualitatively/quantitatively without directly contacting the sample during the entire process and to reduce the wastes.

### SUMMARY OF THE INVENTION

In view of the prior art drawbacks, one aspect of the present invention is to provide an apparatus and a method for quantitatively analyzing an enzyme inhibitor easily and safely.

Another aspect of the present invention is that the test reagents are provided in dry form and each has an amount for one time use only so as to eliminate the need of preparing lots of test solutions.

A further aspect of the present invention is to provide an apparatus for quantitatively analyzing an enzyme inhibitor, which determines the concentration of pesticide remained in a product by determining the amount of the enzyme inhibitor presented in the remained pesticide based on the calibration parameters established by reference samples of known concentrations.

In one embodiment, an apparatus for quantitatively analyzing an enzyme inhibitor, includes a memory unit for storing at least one set of parameters; an interface for receiving a test strip; a reflective type optical detection unit for providing a light beam onto the test strip to obtain a test signal; a processor for processing the test signal based on the parameters to obtain a quantitative analysis result; and a display unit for outputting the quantitative analysis result.

In an exemplary embodiment, the at least one set of parameters includes a set of calibration parameters for determining the relationship between a concentration of the enzyme inhibitor and the test signal. The at least one set of parameters may further include a set of time compensation parameters. In another embodiment, the apparatus may include a thermal sensor for detecting an ambient temperature, and the at least one set of parameters may include a set of temperature compensation parameters. The processor may process the test signal based on the calibration parameters, the time compensation parameters, and the temperature compensation parameters to obtain the quantitative analysis result.

The apparatus may also include a transmission port coupling with the processor to serve as a data transmission interface with an external electronic device to transmit/receive, for example, the test parameters or the quantitative result. In another embodiment, the apparatus may include an amplifier and an analog/digital converter for processing the test signal prior to the processor.

The apparatus may also include a fixture, which is provided to accommodate the test strip so as to prevent the use to directly contact the test strip during the reaction time, to easily place the test strip in an appropriate position, to ensure the contact between the test pads. That is, the test strip can be disposed in the fixture, and the interface is configured to receive the fixture.

In another embodiment, a method for quantitatively analyzing an enzyme inhibitor by the detection apparatus described above, which determines the inhibition of the enzyme based on the enzyme dynamics.

The method includes providing a liquid sample to an enzyme on a test strip to react with the enzyme; folding the test strip to react the enzyme with a color indicator on the test strip; and disposing the test strip in the detection apparatus, wherein the reflective type optical detection unit provides a light beam onto the test strip to obtain a test signal, the processor processes the test signal based on the at least one set of parameters to obtain a quantitative analysis result, and the display unit outputs the quantitative analysis result.

Before the test of an unknown sample, the at least one set of parameters is established by using a reference test strip. The method further includes establishing a set of temperature compensation parameters or a set of time compensation parameters by reference test strips at different temperatures or at different times and storing the set of temperature compensation parameters or time compensation parameters in the memory unit prior to analyzing the test strip. The method further includes calibrating the detection apparatus by a calibration test strip of a given color. In an exemplary embodiment, the processor processes the test signal based on the at least one set of parameters by interpolation. In another embodiment, the processor obtains a percentage of the enzyme inhibitor (Pei) by an equation, Pei=(ΔRef-b−ΔRef-s)/ΔRef-b×100%, wherein ΔRef-b is a reference test signal of a blank test strip and ΔRef-s is the test signal of the sample.

### BRIEF DESCRIPTION OF THE DRAWINGS

FIG. 1A illustrates a schematic view of a fixture and a test strip at an unfolded configuration in accordance with one embodiment of the present invention;

FIG. 1B illustrates a schematic view of FIG. 1 when the test strip disposed on the fixture is at an folded configuration;

FIG. 2A illustrates a perspective view of a detection apparatus in accordance with one embodiment of the present invention;

FIG. 2B illustrates a system diagram of a detection apparatus in accordance with one embodiment of the present invention;

FIG. 3 illustrates a schematic diagram of a calibration curve in accordance with one embodiment of the present invention;

FIG. 4 illustrates a flow chart of a method in accordance with one embodiment of the present invention; and

FIG. 5 illustrates a flow chart of a method in accordance with another embodiment of the present invention.

### DETAILED DESCRIPTION OF THE INVENTION

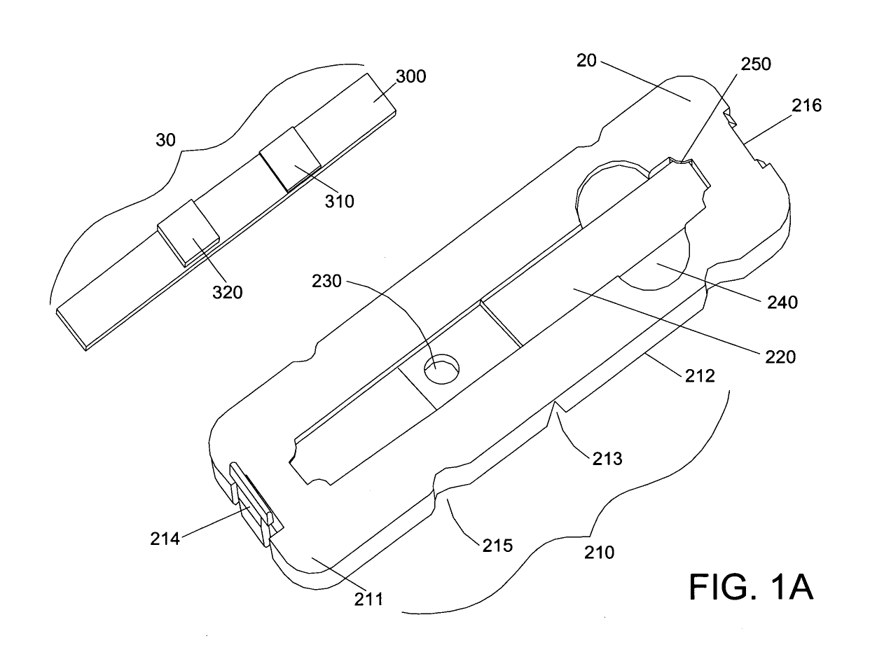

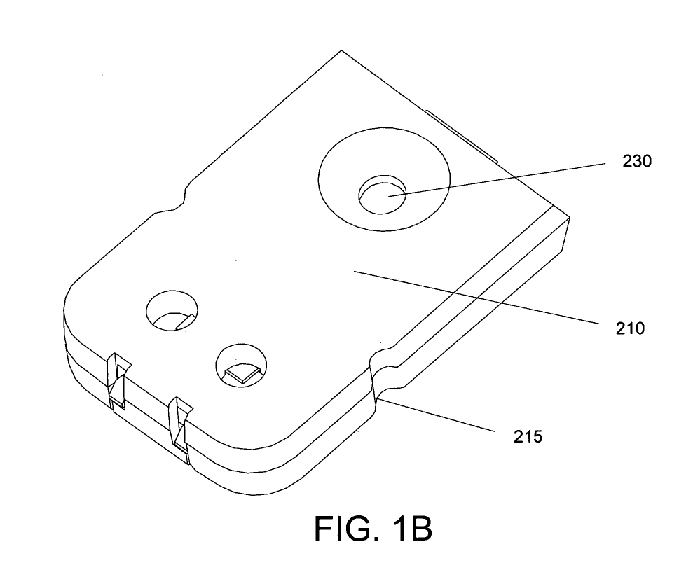

As described above, the present invention provides an apparatus and a method for quantitatively analyzing an enzyme inhibitor, which enable a user to obtain a quantitative result quickly and safely. The present invention may best be understood by reference to the following description in conjunction with the accompanying drawings from FIG. 1 to FIG. 5.

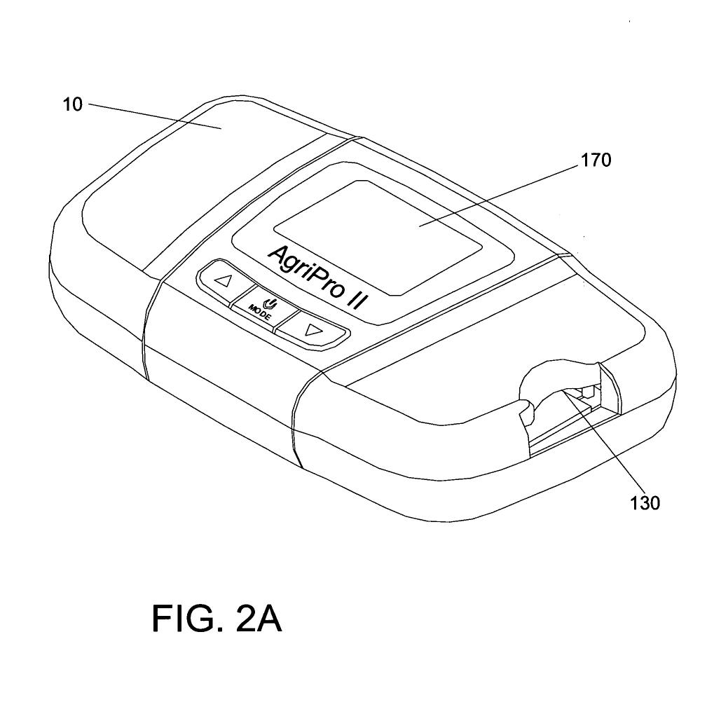

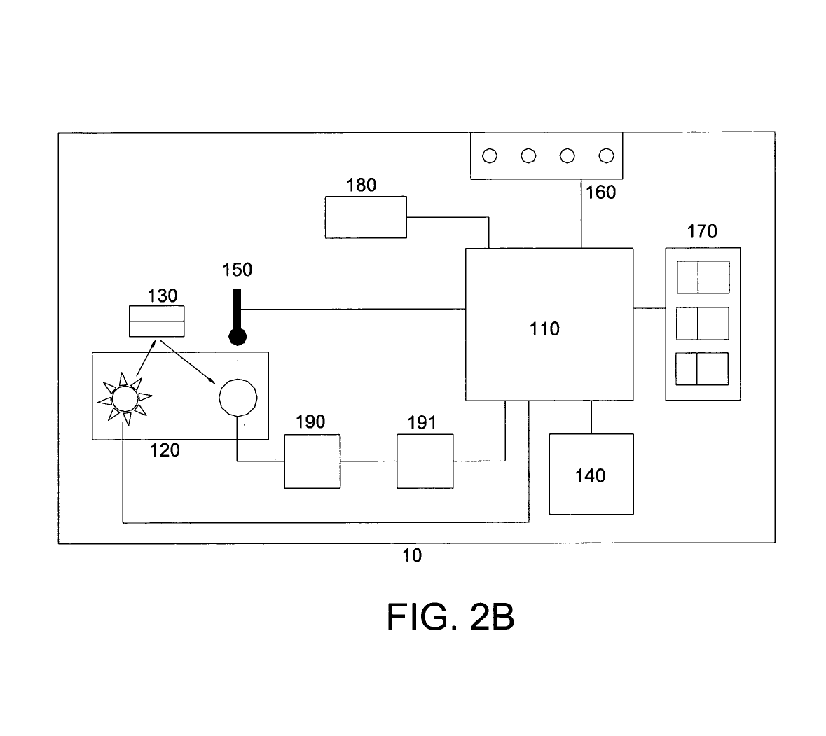

Referring to FIG. 1A, in one embodiment, the present invention provides a detection apparatus 10 (see FIGS. 2A and 2B) and a test strip 30 for analyzing an enzyme inhibitor. The test strip 30 includes a substrate 300, an enzyme pad 310, and a color indicator pad 320. The enzyme pad 310 contains an amount of enzyme for one time use. The color indicator pad 320 contains an amount of color indicator for one time use. The enzyme pad 310 and the color indicator pad 320 are disposed on two opposite sides of the substrate 300. The substrate 300 may be a transparent substrate or a translucent substrate. The test strip 30 is characterized in that the catalytic ability of the enzyme will be inhibited by an enzyme inhibitor, and therefore, the amount of the enzyme can be determined from the inhibitive behavior of the enzyme (i.e. the slowdown of the reaction). The detection apparatus 10 (see FIGS. 2A and 2B) provides a light beam of a specific wavelength, so that the slowdown of the reaction can be determined by the reflected light beam, and the amount of the enzyme inhibitor can be obtained.

In one embodiment, the test strip 30 is preferably disposed in a fixture 20. The fixture includes a casing 210, a groove 220, and a test window 230. The casing 210 includes a first part 211 and a second part 212, which are connected via a flexible part 213 so that the casing 210 can be selectively in an unfolded configuration and a folded configuration. The groove 120 is formed in the casing 210 and configured to receive a test strip, such as the test strip 30. The test window 230 is arranged in the groove 220. When the casing 210 is in the unfolded configuration, a user may dispose/withdraw the test strip 30 through the space provided by a recess 240, as shown in FIG. 1A. When the test strip 30 is disposed in the groove 220, fixing mechanism 250, for example, openings arranged around the corners of the groove 220, is provided to position the test strip 30. When the casing 210 is in the folded configuration (as shown in FIG. 1B), the enzyme pad 310 touches and reacts with the color indicator pad 320, and the detection apparatus 10 may analyze the test strip 30 through the test window 230. Furthermore, the casing 210 may include a first engaging mechanism 214 and a second engaging mechanism 216 for maintaining the casing 210 folded. The first engaging mechanism 214 and the second engaging mechanism 216 can be embodied as a slot and a corresponding protrusion. When a liquid sample is provided to the enzyme pad 310, by deforming the flexible part 213, the first part 211 and the second part 212 of the casing 210 can be folded so as to have the enzyme pad 310 and the color indicator pad 320 closely contacted (as shown in FIG. 1B). Therefore, the enzyme pad 310 can react with the color indicator pad 320. Then, the user may safely and simply insert the fixture 20 including the test strip 30 into the detection apparatus 10 for analysis.

Referring to FIGS. 2A and 2B, in one embodiment, the present invention provides a detection apparatus 10. FIG. 2A illustrates a perspective view of the detection apparatus 10, and FIG. 2B illustrates a system diagram of the detection apparatus 10. As shown in the drawings, the detection apparatus 10 includes a processor 110, a reflective type optical unit 120, an interface 130, a memory unit 140, a thermal sensor 150, a transmission port 160, a display unit 170, a power supply 180, an amplifier 190, and an analog/digital (A/D) converter 191. The power supply 180 provides the detection apparatus 10 required operation power. The memory unit 140 includes, but not limited to, EPROM and is coupled with the process or 110. The memory unit 140 is configured to store one or more than one set of parameters for the processor 110 to use during the analysis. The parameters can be a set of calibration parameters established by calibration test strips of different known concentrations of the enzyme inhibitor. The parameters may further include a set of time compensation parameters or/and a set of temperature compensation parameters.

The interface 130 is configured to receive the fixture 20. For example, the interface 30 can be a slot in the casing 210 of the detection apparatus 10 and is designed to receive the fixture 20 including the test strip 30 or any other calibration test strip or reference test strip. In other words, the interface 130 is designed to accommodate the fixture 20 and position the fixture 20 through a recess 215 (see FIG. 1B). Please note that the interface 130 may have different design to receive other fixtures or test strips. The reflective type optical unit 120 includes a light source and a detector. The light source is configured to provide a light beam of a given wave length onto the test strip 30, while the detector is configured to receive a reflected light beam from the test strip 30 and convert the reflective light beam into a test signal, such as an electrical signal. The test signal is associated with the optical characteristic of the test strip 30. The test signal is then sent to the processor 110 after the amplifier 190 and the A/D converter 191 pre-processing. The processor 110 processes the test signal to obtain a quantitative analysis result based on the parameters stored in the memory unit 140. The display unit 170, such as a liquid crystal display (LCD) or a light emitting display (LED), is coupled with the processor 110 and configured to output the quantitative analysis result. Please note that the display unit 170 may display an instruction of next step, time, date and so on to remind the user the operation procedure.

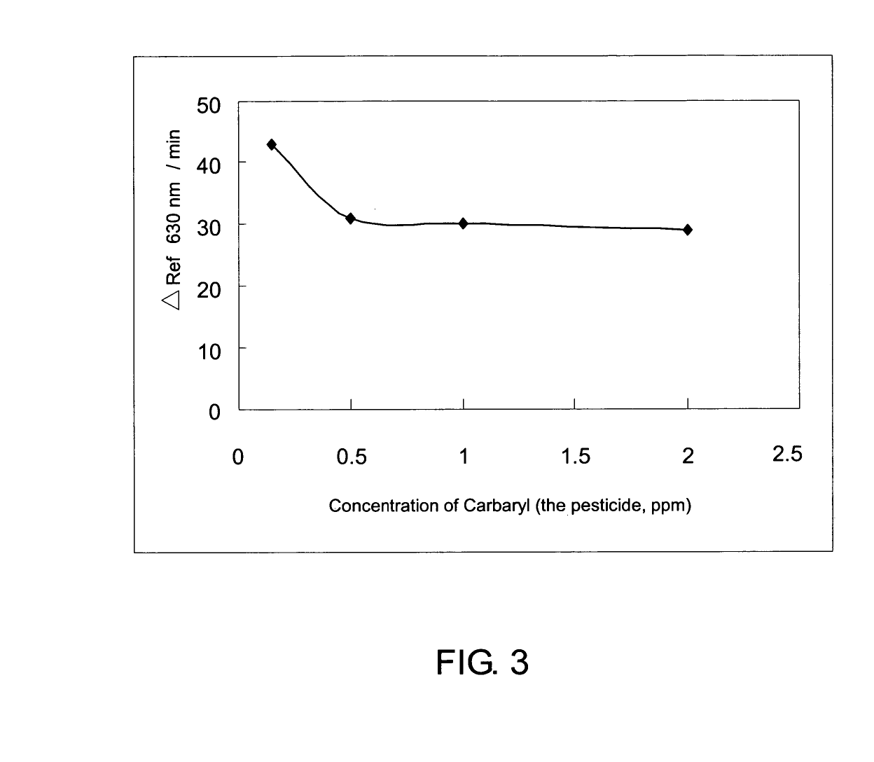

In one embodiment, the parameters include a set of calibration parameters, which determining the relationship between the concentration of the enzyme inhibitor and the test signal. FIG. 3 illustrates a calibration curve of multiple given concentrations of the enzyme inhibitor at the given wavelength within a given time period. For example, a given reference sample of known concentration is tested at the give wavelength, such as 630 nm, within a predetermined time period to obtain a reference signal (ΔRef-STD). After repeating the steps of testing reference samples of different known concentrations, the calibration curve determining the relationship between the concentration of the enzyme inhibitor and the test signal can be established. FIG. 3 shows the relationship between the concentration of pesticide and the test signal. When an unknown sample is tested, the processor 110 processes the test signal based on the calibration curve by interpolation so as to determine the concentration of enzyme inhibitor in the unknown sample. Please note that different calibration curves can be established according to the type of enzyme inhibitor, concentration, temperature, time period, and so on. Moreover, the parameters stored in the memory unit 140 can be updated according to the user's need.

In another embodiment, the thermal sensor 150 is configured to detect the ambient temperature during the analysis, and the parameters further include a set of temperature compensation parameters. In such an arrangement, the processor 110 may process the test signal based on the temperature compensation parameters. For example, the present invention may be implemented to provide multiple sets of temperature compensation parameters at different temperatures for reference samples of different given concentrations. Accordingly, even the temperature may affect the activity of enzyme inhibitor, the present invention still can obtain an accurate test result based on the temperature compensation parameters.

Similarly, in another embodiment, the parameters may include a set of time compensation parameters based on different reaction time for reference samples of same or different given concentrations. Accordingly, even the reaction time is different, the processor 110 can obtain an accurate test result based on the time compensation parameters.

Moreover, the transmission port 160 is provided to couple with the processor 110 and serve as a data transmission interface with an external electronic device. For example, the transmission port 160 may include, but not limited to, RS232 or USB, and transmit/receive, for example, the test result or the test parameters from a computer.

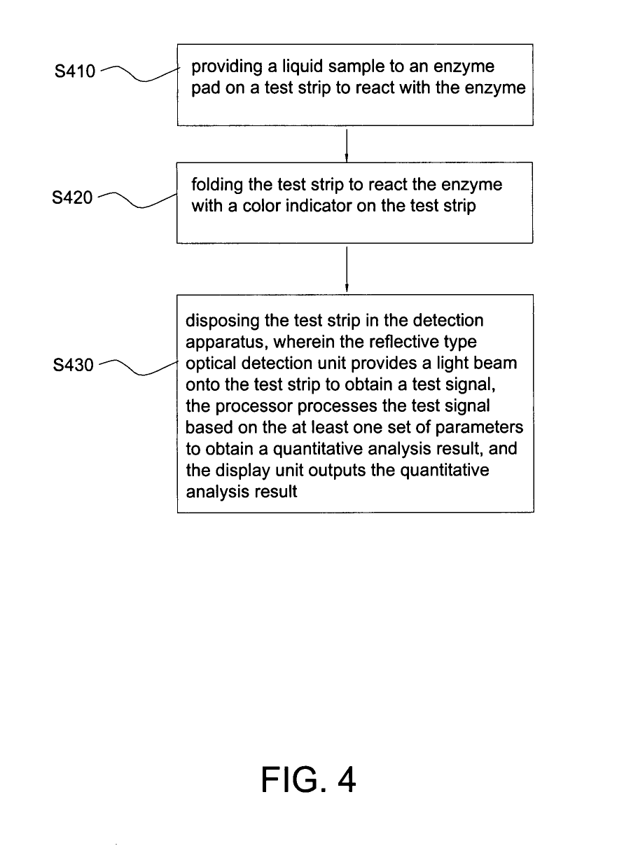

With reference to FIG. 4, in another embodiment, the present invention also provides a method for quantitatively analyzing the enzyme detector by a detection apparatus, such as the detection apparatus 20 shown in FIGS. 2A and 2B. The method includes providing a liquid sample to an enzyme pad on a test strip to react with the enzyme (S410); folding the test strip to react the enzyme with a color indicator on the test strip (S420); and disposing the test strip in the detection apparatus (S430), wherein the reflective type optical detection unit provides a light beam onto the test strip to obtain a test signal, the processor processes the test signal based on the at least one set of parameters to obtain a quantitative analysis result, and the display unit outputs the quantitative analysis result.

In another embodiment, the method further includes transmitting the quantitative analysis result to an external electronic device, such as a computer. Moreover, prior to analyzing the test strip, the at least one set of parameters is established by using at least one reference test strip. Optionally, the method includes the steps of establishing a set of temperature compensation parameters or time compensation parameters by reference test strips at different temperatures within different time periods and storing the set of temperature compensation parameters or time compensation parameters in the memory unit prior to analyzing the test strip. Then, the processor may process the test signal based on the stored calibration, time compensation, and/or the temperature compensation parameters. Moreover, the method may include a step of calibrating the detection apparatus by a calibration test strip of a given color. That is, a fixture of a given color or a fixture with a reference color strip therein may be applied to calibrate the detection apparatus to inspect whether the reflective type optical unit functions normally. For example, the detection apparatus shall output same result for a reference color strip every time when the reference color strip is applied. If the detection apparatus shows a color calibration result different from the last time calibration, it indicates that the detection apparatus is not in a normal operation status. Furthermore, the processor processes the test signal based on the at least one set of parameters by interpolation. Alternatively, in another embodiment, the processor obtains an inhibition percentage of the enzyme inhibitor (Pei) by an equation, Pei=(ΔRef-b−ΔRef-s)/ΔRef-b×100%, wherein ΔRef-b is a reference test signal of a blank test strip and ΔRef-s is the test signal of the sample.

The present invention provides a simple and easy operation apparatus and methods for quantitatively analyzing the enzyme inhibitor based on the feature of pesticide inhibiting the activity of enzyme, so that the amount of pesticide remained in a product can be determined. The present invention can be implemented to detect any pesticide with the enzyme inhibiting characteristic, which includes but not limited to organophosphorus, carbamate or thio-organophosphorus treated by a bromide water.

The test parameters utilized in the present invention may include unit conversion parameters, calibration parameters, time compensation parameters, temperature compensation parameters, available test temperature range, etc.

In the following example, specific chemicals and reactions are adopted to exemplarily explain the concept of the present invention, however, please note that the present invention is not limited to the chemicals or the reaction recited in the example.

The chemicals are listed below:

Enzyme: Acetylcholinesterase (AChE)

Color Indicator: Indoxyl acetate

Compound: Indophenol

Under the catalysis of acetylcholinesterase, indoxyl acetate is hydrolyzed into acetic acid and indophenol. The catalysis activity will be strongly suppressed by pesticides such as organophosphorus and carbamate. Accordingly, the pesticides remained in the samples can be determined by observing the variations of the absorption peak at the visible region, for example at 630 nm.

The method includes a step of providing an enzyme, such as AChE, a color indicator, such as indoxyl acetate. The enzyme and the color indicator of the present invention are provided in dray form, such as a dry pad form, and for one time use only. For example, a water-insoluble substrate, such as a plastic substrate, is provided, and the enzyme pad and the color indicator pad are disposed on two opposite sides of the substrate as a test strip. Then, the test strip can be disposed in the fixture, and a liquid sample is provided to the enzyme pad to react with the enzyme. The fixture is folded to tightly contact the enzyme pad with the color indicator pad so that the sampled enzyme is reacted with the color indicator.

Moreover, other chemical reagents may be used in the present invention in accordance with the design need. For example, a buffer salt may be used to adjust PH value of the liquid sample.

A reference sample with a known amount of enzyme inhibitor is provided to a test strip. For example, a reference sample without enzyme inhibitor (amount=0) is provided to a test strip as a standard sample strip, which will be detected by the detection apparatus.

By using the reflective type optical unit to emit a light beam of specific wavelength (e.g. 630 nm), a reflected light signal from the standard strip is measured as a reference signal (ΔRef-b) within a predetermined time period. Multiple reference samples with different known amount of enzyme inhibitor can be subsequently detected to establish a calibration curve for determining the relationship between the concentration of the enzyme inhibitor and the test signal. FIG. 3 shows an exemplary calibration curve.

A sample of unknown amount of enzyme inhibitor is provided, which is referred to a test sample. The test sample is provided to an enzyme pad of a test strip in a fixture, and the detection operation is repeated to obtain a test signal (ΔRef-s). For example, the reflected type optical unit emits a light beam of the specific wavelength to the test strip and receives a reflected light signal as the test signal. Comparing the test signal with the calibration curve, the amount of the enzyme inhibitor presented in the test sample can be determined.

For example, according to the calibration curve shown in FIG. 3, the processor may process the test signal by interpolation to obtain the concentration of the enzyme inhibitor in the test sample. In another embodiment, the processor may obtains an inhibition percentage of the enzyme inhibitor (Pei) by the equation, Pei=(ΔRef-b−ΔRef-s)/ΔRef-b×100%, wherein ΔRef-b is a reference test signal of a blank test strip and ΔRef-s is the test signal of the sample. The blank test strip is referred to the reference sample without enzyme inhibitor as described above.

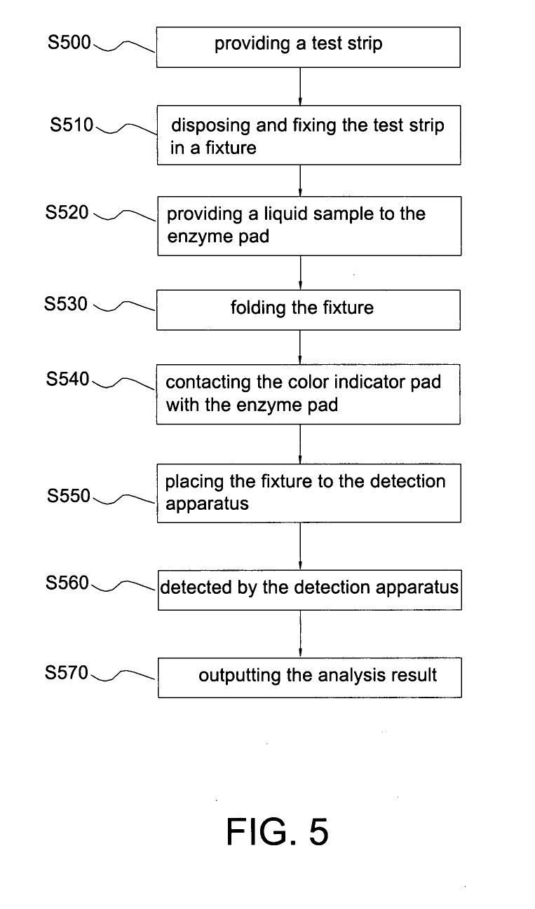

FIG. 5 illustrates a flow chart of a method in accordance with another embodiment of the present invention. First, at S500, a test strip is provided, which can be a test strip with the enzyme pad the color indicator pad on opposite sides of a substrate, as described above. At S510, the test strip is disposed and fixed in a fixture. At S520, an unknown pesticide liquid sample or a blank reference sample is provided to the enzyme pad. At S530, the fixture is folded, and the color indicator pad and the enzyme pad are closely contacted to react with each other (S540). At S550, the fixture is inserted into the detection apparatus. At S560, the reflected light signal (ΔRef-s) is determined within a time interval of 1 minute. Then at S570, the display unit output the analysis result to the user, which may include the concentration of the enzyme inhibitor or the inhibition percentage of the enzyme inhibitor, the operation date or time, etc.

In other words, the present invention provide an apparatus and a method for quantitatively analyzing an enzyme inhibitor easily and safely, so that the user is prevented to directly contact the test strip during the operation. Furthermore, the fixture can be repeatedly utilized so as to reduce the wastes, and the reaction reagents are provided in dry form for one time use so as to simplify the waste solution treatment.

The present invention has been described above with reference to preferred embodiments. However, those skilled in the art will understand that the scope of the present invention need not be limited to the disclosed preferred embodiments. On the contrary, it is intended to cover various modifications and equivalent arrangements within the scope defined in the following appended claims. The scope of the claims should be accorded the broadest interpretation so as to encompass all such modifications and equivalent arrangements.

What is claimed is:

1. An apparatus for quantitatively analyzing an enzyme inhibitor, comprising:
a memory unit for storing at least one set of parameters;
an interface for receiving a test strip;
a reflective type optical detection unit for providing a light beam onto the test strip to obtain a test signal;
a processor for processing the test signal based on the parameters to obtain a quantitative analysis result; and
a display unit for outputting the quantitative analysis result.

2. The apparatus of claim 1, wherein the at least one set of parameters comprises a set of calibration parameters for determining the relationship between a concentration of the enzyme inhibitor and the test signal.

3. The apparatus of claim 2, wherein the at least one set of parameters further comprises a set of time compensation parameters.

4. The apparatus of claim 2, further comprising a thermal sensor for detecting an ambient temperature, wherein the at least one set of parameters further comprises a set of temperature compensation parameters.

5. The apparatus of claim 1, further comprising a transmission port coupling with the processor serving as a data transmission interface with an external electronic device.

6. The apparatus of claim 1, further comprising an amplifier and an analog/digital converter for processing the test signal prior to the processor.

7. The apparatus of claim 1, wherein the test strip is disposed in a fixture, and the interface is configured to receive the fixture.

8. A method for quantitatively analyzing an enzyme inhibitor by a detection apparatus, the detection apparatus comprising a processor, a memory unit for storing at least one set of parameters, a reflective type optical detection unit, the method comprising:
providing a liquid sample to an enzyme pad on a test strip to react with the enzyme;
folding the test strip to react the enzyme with a color indicator on the test strip; and
disposing the test strip in the detection apparatus,
wherein the reflective type optical detection unit provides a light beam onto the test strip to obtain a test signal, the processor processes the test signal based on the at least one set of parameters to obtain a quantitative analysis result, and the display unit outputs the quantitative analysis result.

9. The method of claim 8, further comprising transmitting the quantitative analysis result to an external electronic device.

10. The method of claim 8, wherein the at least one set of parameters is established by using a reference test strip prior to analyzing the test strip.

11. The method of claim 8, further comprising establishing a set of temperature compensation parameters by reference test strips at different temperatures and storing the set of temperature compensation parameters in the memory unit prior to analyzing the test strip.

12. The method of claim 8, further comprising establishing a set of time compensation parameters by reference test strips at different time and storing the set of time compensation parameters in the memory unit prior to analyzing the test strip.

13. The method of claim 8, further comprising calibrating the detection apparatus by a calibration test strip of a given color.

14. The method of claim 8, wherein the processor processes the test signal based on the at least one set of parameters by interpolation.

15. The method of claim 8, wherein the processor obtains an inhibition percentage of the enzyme inhibitor (Pei) by an equation, Pei=(ΔRef-b−ΔRef-s)/ΔRef-b×100%, wherein ΔRef-b is a reference test signal of a blank test strip and ΔRef-s is the test signal of the sample.
